(19) United States
(12) Patent Application Publication
(10) Pub. No.: US 2012/0330376 A1
(43) Pub. Date: Dec. 27, 2012

(54) SYSTEMS AND METHODS FOR MAKING AND USING ELECTRICAL STIMULATION SYSTEMS FOR PROVIDING THERAPY TO LARGE ANIMALS

(75) Inventors: Daniel Vincent Flynn, Charlottesville, VA (US); Gary Alan Owings, Phoenix, MD (US)

(73) Assignee: Equine OrthoCare, LLC, Phoenix, MD (US)

(21) Appl. No.: 13/169,460

(22) Filed: Jun. 27, 2011

(51) Int. Cl. A61D 99/00; A61N 1/36

(52) U.S. Cl. 607/52

(57) ABSTRACT

An electrical stimulation system for providing non-invasive therapeutic stimulation to a large animal includes a surcingle for placement around a girth of the large animal. The surcingle includes a non-stretchable first element and a stretchable second element. Foam wedges are disposed on the first element for flanking a spine of the large animal during stimulation. A control module with a signal generator is coupleable to the first element. A treatment electrode is disposed on a lead that is electrically coupleable to the control module. The treatment electrode has a conductive inner surface for disposing over skin of the large animal at a target stimulation location. The treatment electrode transmits to the target stimulation location electrical signals received from the signal generator. A return electrode disposed on the first element is electrically coupled to the control module and receives electrical signals output from the treatment electrode.

### FIELD

The present invention is directed to the area of electrical stimulation systems for use with large animals and methods of making and using the systems. The present invention is also directed to electrical stimulation systems for providing orthopedic therapy to large animals.

### BACKGROUND

Electrical stimulation systems for large animals (e.g., horses, oxen, mules, burrows, donkeys, camels, gnus, yaks, and the like) have proven therapeutic in a variety of disorders. For example, electrical stimulation systems have been used as a therapeutic modality for the treatment of a variety of orthopedic conditions, including osteoarthritis with attending tendon and ligament injury and non-union fracture repair.

An electrical stimulation system can include a control module (with a signal generator), one or more treatment electrodes, and one or more return electrodes. The signal generator generates electrical signals that are delivered by the one or more treatment electrodes to a target stimulation location on a large animal. The one or more return electrodes draw current transmitted from the one or more treatment electrodes through a portion of the large animal such that the current passes in proximity to an adversely-affected region of the large animal.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an electrical stimulation system suitable for providing therapy to a large animal, according to the invention;

FIG. 2 is a schematic view of another embodiment of the electrical stimulation system of FIG. 1, according to the invention;

FIG. 3A is a schematic bottom view of one embodiment of an inner surface of a surcingle suitable for use with the system of FIGS. 1 and 2, the surcingle including a first element and a second element, according to the invention;

FIG. 3B is a schematic top view of one embodiment of an outer side of the surcingle of FIG. 3A, according to the invention;

FIG. 3C is a schematic side view of one embodiment of a side of the surcingle of FIG. 3A, according to the invention;

FIG. 4A is a schematic side view of one embodiment of the electrical stimulation system of FIGS. 1 and 2 and the surcingle of FIGS. 3A-3C disposed on a horse, the electrical stimulation system including a single treatment electrode, according to the invention;

FIG. 4B is a schematic side view of another embodiment of the electrical stimulation system of FIGS. 1 and 2 and the surcingle of FIGS. 3A-3C disposed on the horse of FIG. 4A, the electrical stimulation system including two treatment electrodes, according to the invention;

FIG. 4C is a schematic side view of yet another embodiment of the electrical stimulation system of FIGS. 1 and 2 and the surcingle of FIGS. 3A-3C disposed on the horse of FIG. 4A, the electrical stimulation system including four treatment electrodes, according to the invention;

FIG. 5 is a schematic side view of another embodiment of the electrical stimulation system of FIGS. 1 and 2 and the surcingle of FIGS. 3A-3C disposed on the horse of FIG. 4A, the electrical stimulation system including a treatment electrode disposed on a neck sleeve, according to the invention;

FIG. 6 is a schematic side view of another embodiment of the electrical stimulation system of FIGS. 1 and 2 and the surcingle of FIGS. 3A-3C disposed on the horse of FIG. 4A, the electrical stimulation system including a treatment electrode disposed on a breast collar, according to the invention;

FIG. 7 is a schematic side view of another embodiment of the electrical stimulation system of FIGS. 1 and 2 and the surcingle of FIGS. 3A-3C disposed on the horse of FIG. 4A, the electrical stimulation system including a treatment electrode disposed on a head hood, according to the invention;

FIG. 8 is a schematic side view of another embodiment of the electrical stimulation system of FIGS. 1 and 2 disposed on the horse of FIG. 4A, the electrical stimulation system including a treatment electrode, a return electrode, and a control module disposed on the head hood of FIG. 7, according to the invention;

FIG. 9 is a schematic side view of another embodiment of the electrical stimulation system of FIGS. 1 and 2 disposed on the horse of FIG. 4A, the electrical stimulation system including a treatment electrode, a return electrode, and a control module disposed on a breeching, according to the invention;

FIG. 10 is a schematic side view of another embodiment of the electrical stimulation system of FIGS. 1 and 2 and the surcingle of FIGS. 3A-3C disposed on the horse of FIG. 4A, the electrical stimulation system including two treatment electrodes disposed on a forelimb of the horse, according to the invention; and

FIG. 11 is a schematic side view of another embodiment of the electrical stimulation system of FIGS. 1 and 2 and the surcingle of FIGS. 3A-3C disposed on the horse of FIG. 4A, the electrical stimulation system including two treatment electrodes disposed on a forelimb of the horse, the two treatment electrodes coupled to each other via an extension lead, according to the invention.

### DETAILED DESCRIPTION

The present invention is directed to the area of electrical stimulation systems for use with large animals and methods of making and using the systems. The present invention is also directed to electrical stimulation systems for providing orthopedic therapy to large animals.

Many large animals (e.g., horses, oxen, mules, burrows, donkeys, camels, gnus, yaks, and the like) suffer from one or more orthopedic conditions, including osteoarthritis, fractures, inflammation, and other connective tissue disorders. Such adverse orthopedic conditions may decrease the large animal's physical performance. Such conditions may also decrease the large animal's quality of life.

Healthy large animal joints, when in use, generate electrical fields. The generated electrical fields may play a role in activating repair and regeneration of joint tissue. When a joint is compromised by one or more orthopedic conditions, the strength of the generated electrical field may be decreased, thereby potentially reducing repair and regeneration of joint tissue. Consequently, the affected joint may not be able to repair itself as fast as it wears out, resulting in a gradual degeneration of the joint.

One method of treatment for adverse orthopedic conditions is electrical stimulation. As herein described, an electrical stimulation system is provided which provides stimulation therapy to a large animal. In at least some embodiments, the system mimics and enhances the electric field generated by the affected tissue (e.g., a joint), thereby increasing the ability of the joint to activate its repair and regeneration mechanisms. In at least some embodiments, the electrical stimulation system is non-invasive.

Suitable electrical stimulation systems include, but are not limited to, a signal generator, one or more treatment electrodes, and one or more return electrodes, where the one or more treatment electrodes and the one or more return electrodes remain external to the large animal during therapy. An example of an electrical stimulation system with a signal generator, a treatment electrode, and a return electrode, where the treatment electrode and the return electrode are disposed external to a human patient is found in, for example, U.S. Pat. No. 5,273,033, which is incorporated by reference.

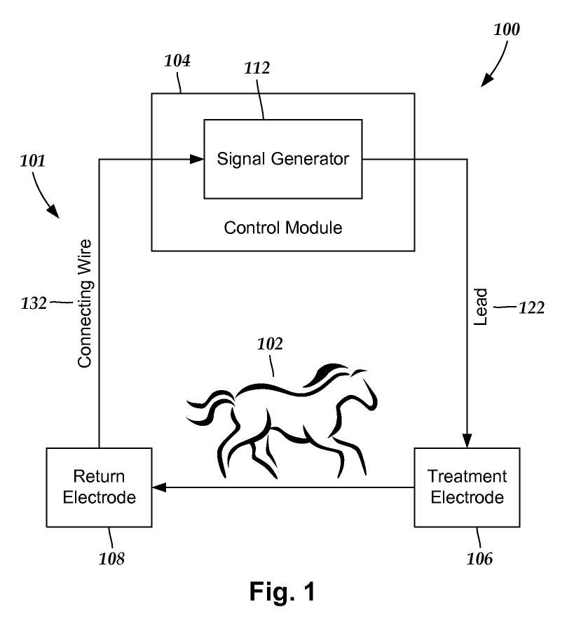

FIG. 1 is a schematic view of one embodiment of an electrical stimulation system (“system”) 100 having a single stimulation circuit 101 suitable for providing therapy to a large animal 102. The system 100 includes a control module 104, a treatment electrode 106, and a return electrode 108. A signal generator 112 is disposed in the control module 104 and is configured to provide therapeutic electrical signals to the treatment electrode 106. The treatment electrode 106 is disposed on a lead 122 that is coupleable with the control module 104 and configured and arranged to transmit the therapeutic electrical signals from the signal generator 112 to the treatment electrode 106. A return wire 132 is configured and arranged to transmit the therapeutic electrical signals from the return electrode 108 to the signal generator 112.

The treatment electrode 106 is configured and arranged for disposing over the skin of the large animal 102 at a target stimulation location. The target stimulation locations can be any suitable location on the large animal 102 including, for example, the jaw, neck, forelimb, hindlimb, knee, fetlock, hock, stifle, shoulder, foot, shin, splint bone, back, hind quarter, or the like or combinations thereof.

The return electrode 108 is configured and arranged to disposing over the skin of the large animal 102 at a location that is remote from the target stimulation location (i.e., the location of the treatment electrode 106). A remote location is at least 10 cm, 20 cm, 30 cm, 40 cm, 50 cm, 60 cm, 70 cm, 80 cm, 90 cm, 100 cm, 120 cm, 140 cm, 160 cm, 180 cm, 200 cm, or more from the target stimulation region. In at least some embodiments, during operation the return electrode 108 is disposed at least 10 cm, and no more than 120 cm, from the treatment electrode 106. In at least some embodiments, during operation the return electrode 108 is disposed at least 10 cm, and no more than 100 cm, from the treatment electrode 106. In at least some embodiments, during operation the return electrode 108 is disposed at least 10 cm, and no more than 80 cm, from the treatment electrode 106.

The return electrode 108 is configured and arranged to draw the therapeutic electrical signals transmitted by the treatment electrode 106, through a portion of the large animal 102 to the return electrode 108. The return electrode 108 can be positioned such that, when the electrical signals are passing from the treatment electrode 106 to the return electrode 108, the electrical signals pass in proximity to adversely-affected tissue(s) (e.g., a joint affected by osteoarthritis, inflammation, a fracture, or the like). The treatment electrode 106 and the return electrode 108 can be formed from any suitable conductive materials. In at least some embodiments, the return electrode 108 is formed from a soft material impregnated with silver. In some cases, a conductive gel may be applied to the electrodes 106, 108 to improve electrical contact between the electrodes 106, 108 and the skin of the large animal 102.

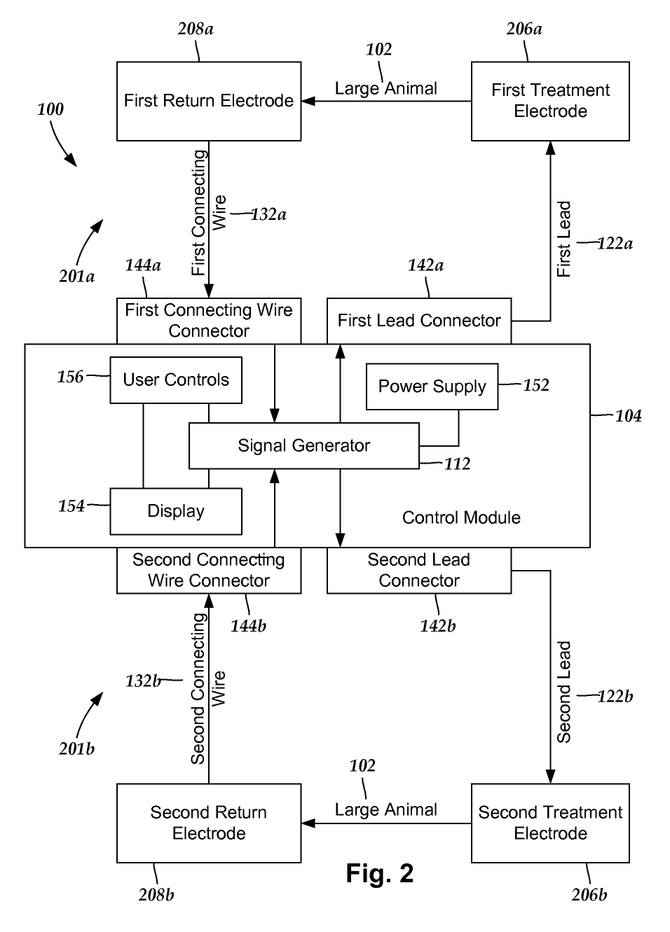

The system 100 shown in FIG. 1 is configured with a single treatment electrode 106 and a single return electrode 108. It will be understood that the system 100 may include any suitable number of treatment electrode 106 and return electrodes 108. FIG. 2 is a schematic view of another embodiment of the system 100 with two stimulation circuits: a first stimulation circuit 201a, and a second stimulation circuit 201b. The first stimulation circuit 210a includes a first treatment electrode 206a, a first return electrode 208a, a first lead 122a, and a first return wire 132a. The second stimulation circuit 210b includes a second treatment electrode 206b, a second return electrode 208b, a second lead 122b, and a second return wire 132b.

The control module 104 includes a first lead connector 142a for coupling to the first lead 122a, a second lead connector 142b for coupling to the second lead 122b, a first return wire connector 144a for coupling to the first return wire 132a, and a second return wire connector 144b for coupling to the second return wire 132b. The control module 104 may also include a power supply 152 (e.g., a 9V battery, or the like), one or more displays 154 (e.g., an LCD display, or the like), and user controls 156 for powering the control module 104 on, adjusting stimulation parameters, or the like. The control module 104 can be formed in any suitable size. In some cases, the control module 104 is sized for disposing on a strap or harness worn by the large animal. Optionally, the control module 104 is approximately the size of a typical hand-held mobile electronic device.

The electrical signals generated by the signal generator 112 can be of any suitable voltage for providing therapy to the large animal 102. In some embodiments, the electrical signals generated by the signal generator 112 have a constant voltage that is no greater than 12 volts. In at least some embodiments, the electrical signals generated by the signal generator 112 are pulsed currents. In some embodiments, the electrical signals are pulsed direct current signals having voltages that are no greater than 50 volts. In other embodiments, the electrical signals are high voltage pulsed currents having short voltage peaks that are no greater than 330 volts.

In the case of pulsed currents, the electrical signals generated by the signal generator 112 may have frequencies that are no greater than 110 Hz. In other cases, such as when high voltage is being used, frequencies may be in the megahertz range. For example, in at least some embodiments, the electrical signals generated by the signal generator 112 have frequencies that are at least 1 MHz, and no greater than 10 MHz.

Optionally, the therapeutic electrical signals generated by the signal generator 112 are negatively-biased. In which case, the treatment electrode(s) can be implemented as anode(s) and the return electrode(s) can be implemented as cathode(s) in order to draw the generated signals through large animal tissue between the treatment electrode(s) and the return electrode(s). It will be understood that, alternately, the electrical signals generated by the signal generator 112 can be positively-biased, or a combination of both positively-biased and negatively-biased components.

In some cases, during treatment of the large animal the signals generated by the system 100 are sub-sensory. In which case, the system 100 can be used without sedating or anesthetizing the large animal. In at least some embodiments, the amplitude of the electrical signals output by the system 100 are increased until the large animal elicits a visibly-detectable response indicating that the large animal can sense (e.g., feel) the electrical signals. The amplitude can then be reduced slightly to an operating level that is sub-sensory during therapy. Optionally, the system 100 can be used with other non-electrical treatment methods (e.g., medications, hand walking, or the like or combinations thereof).

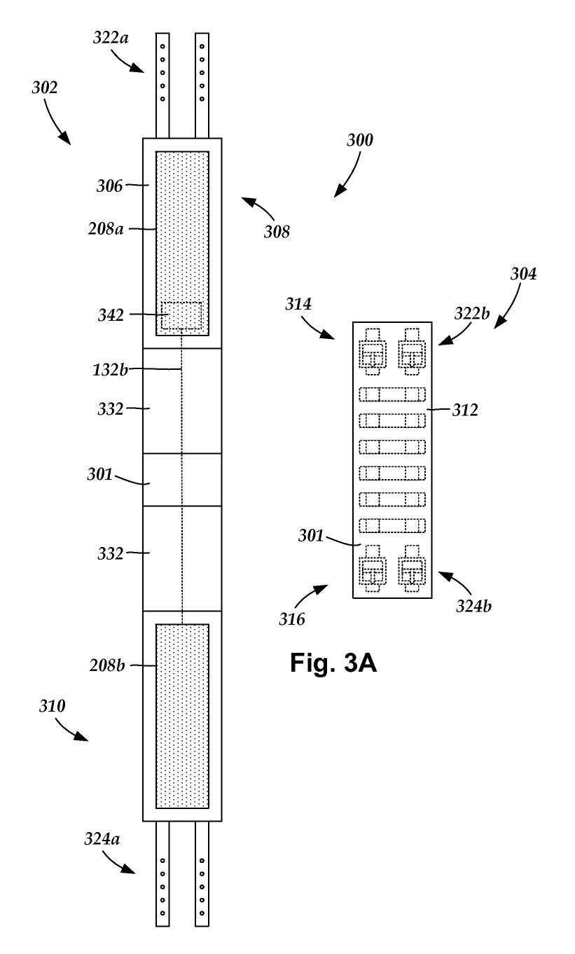

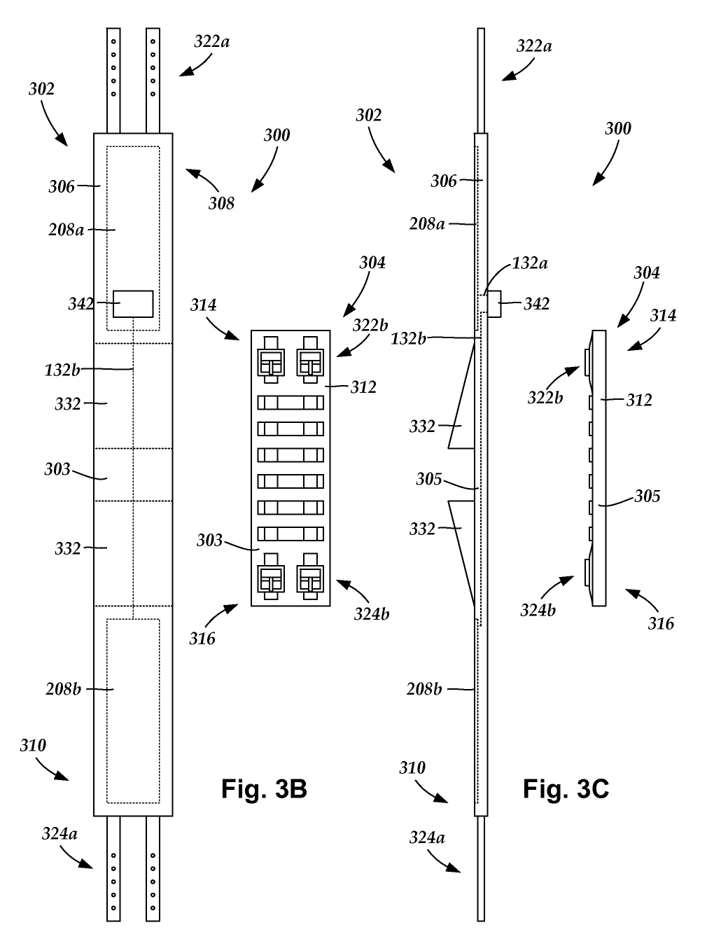

The control module, return electrode(s), and treatment electrode(s) can be disposed on the large animal at any suitable location including, for example, a surcingle, a head hood, a neck sleeve, a breast collar, a crupper, a breeching, a saddle, or the like or combinations thereof. In some cases, the control module is disposed on a surcingle. FIG. 3A is a schematic bottom view of one embodiment of an inner (bottom) surface of a surcingle 300 suitable for holding the control module 104. In at least some embodiments, the surcingle 300 is also configured and arranged to receive one or more of the return electrodes 208a and 208b. FIG. 3B is a schematic top view of one embodiment of an outer (top) surface 303 of the surcingle 300. FIG. 3C is a schematic side view of one embodiment of a side 305 of the surcingle 300.

The surcingle 300 includes an elongated first element 302 and an elongated second element 304. The first element 302 includes a body 306, a first end 308, and an opposing second end 310. The second element 304 includes a body 312, a first end 314, and an opposing second end 316. In preferred embodiments, the length of the first element 302 (i.e., the distance between the first end 308 and the second end 310) is larger than the length of the second element 304 (i.e., the distance between the first end 314 and the second end 316)

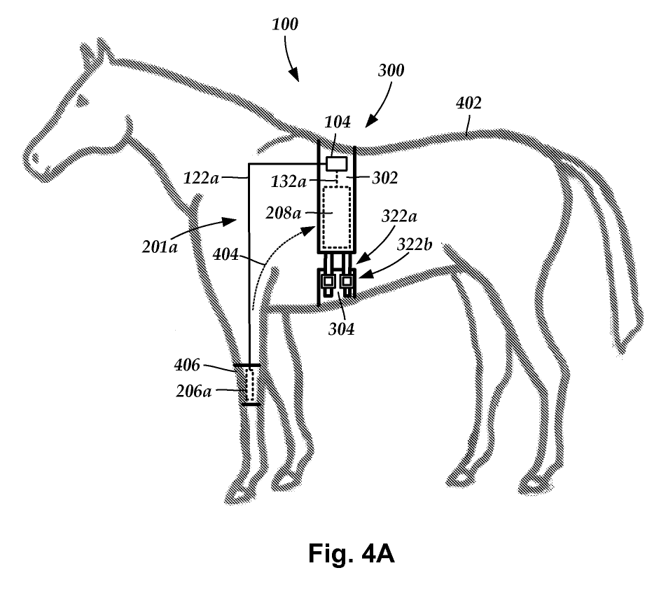

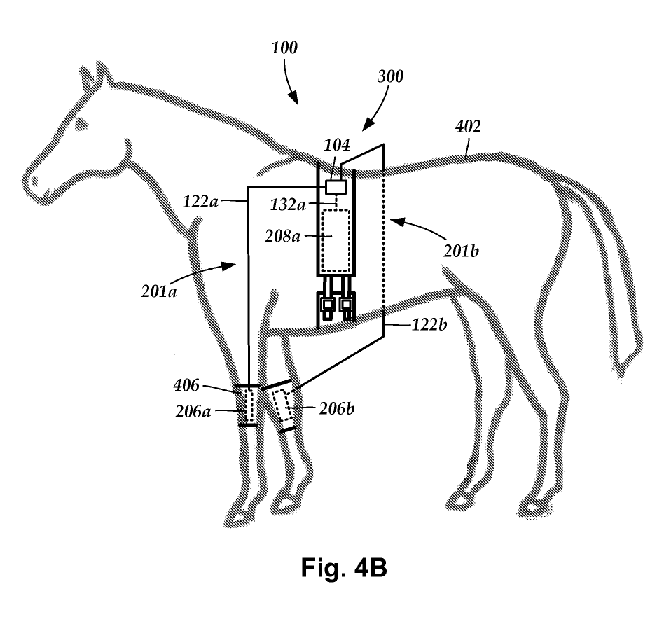

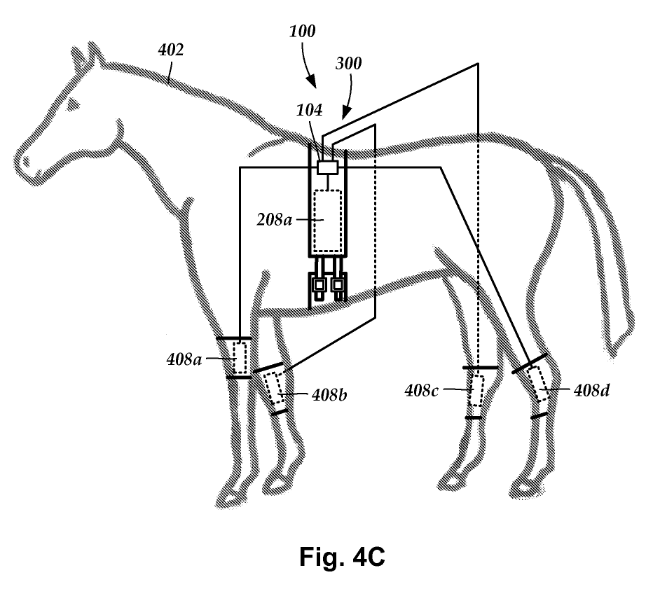

The body 306 of the first element 302 and the body 312 of the second element 304 are configured and arranged to couple together end-to-end such that the first element 302 and the second element 304 collectively have a length that is sufficiently-long to fit around a girth (e.g., the belly and back) of the large animal 102. In preferred embodiments, the surcingle 300 is configured and arranged for disposing around the girth of the large animal 102 such that the first element 302 is disposed over the back of the large animal 102 and the second element 304 is disposed over the belly of the large animal 102 (see e.g., FIG. 4A).

In at least some embodiments, the first end 308 of the first element 302 includes a first end coupler 322a, such as one or more straps, configured and arranged to fasten to a corresponding first end coupler 322b, such as one or more buckles, disposed on the second element 304. Similarly, in at least some embodiments the second end 310 of the first element 302 includes a second end coupler 324a, such as one or more straps, configured and arranged to fasten against a corresponding second end coupler 324b, such as one or more buckles, disposed on the second element 304. It will be understood that the straps and the buckles can be disposed on the first element 302 and the second element 304 in any interconnectable combination. In alternate embodiments, other types of couplers may be used in lieu of, or in addition to, one or more straps and corresponding buckles including, for example, hook and loop fasteners, interlocking features, snaps, or the like or combinations thereof.

In FIGS. 3A-3C, the surcingle 300 is shown having couplers that each include two straps and two corresponding buckles on each end of the first and second elements. It will be understood that buckle-and-strap couplers may include any suitable number of buckles and straps. It may be advantageous to include a plurality of buckles and straps on each end of the first and second elements for a sturdy coupling, and also to enable adjustment of the fit of the surcingle 300 around the girth of the large animal 102 when the surcingle 300 is disposed on the large animal 102.

As best shown in FIG. 3A, in at least some embodiments the first return electrode 208a and the second return electrode 208b are disposed on the inner surface 301 of the first element 302. The first return electrode 208a and the second return electrode 208b are configured and arranged such that, when the surcingle 300 is fastened onto the girth of the large animal 102, inner surfaces of the first return electrode 208a and the second return electrode 208b contact the skin of the large animal 102. In at least some embodiments, the first return electrode 208a and the second return electrode 208b are disposed on the inner surface 301 of the first element 302 such that the first return electrode 208a and the second return electrode 208b are disposed against opposing sides (e.g., right and left) of the large animal 102 when the surcingle 300 is fastened onto the girth of the large animal 102.

The surcingle 300 can be formed from any suitable materials. In at least some embodiments, the first element 302 and the second element 304 are formed from the same material (e.g., leather, ripstop nylon, neoprene, or the like or combinations thereof).

In at least some embodiments, the first element 302 is formed from one or more non-stretchable materials (e.g., leather, ripstop nylon, or the like) and the second element 304 is formed from one or more elastic materials (e.g., neoprene, or the like) that make the second element 304 more stretchable than the first element 302.

It may be advantageous to form the first element 102 from a non-stretchable material so that the first element 302 maintains a snug fit of the surcingle 300 around the girth of the large animal 102 when the surcingle 300 is disposed on the large animal 102 and does not move relative to the large animal. Movement of the surcingle 300 relative to the large animal 102 may adversely affect therapy by changing the directionality of flow of the electrical signals through the large animal 102.

Additionally, it may be advantageous to form the first element 102 from a non-stretchable material because the return electrode(s) may not operate properly when disposed on elastic material. Stretching of the first element 302 when one or more return electrodes are disposed thereon may also potentially change the directionality of flow of the electrical signals through the large animal 102 and adversely affect electrical contact between the return electrode(s) and the skin of the large animal 102. Thus, it may be advantageous to form the first element 102 from a non-stretchable material to maintain constant and consistent contact between the skin of the large animal 102 and each of the first return electrode 208a and the second return electrode 208b.

It may be advantageous to form the second element 304 from an elastic material to enable the surcingle 300 to form a snug, yet non-constricting fit around the girth of the large animal 102 when the surcingle 300 is disposed around the large animal 102, especially when the first element 302 is formed from a non-stretchable material.

In at least some embodiments, one or more pads 332 are disposed along the inner surface 301 of the first element 302. The one or more pads 332 can be used to reduce sliding of the surcingle 300 relative to the large animal 102 when the surcingle 300 is disposed on the large animal 102. The one or more pads 332 can also be used to prevent rubbing of the surcingle 300 against the spine of the large animal 102 when the surcingle 300 is disposed around the girth of the large animal 102. In at least some embodiments, the surcingle 300 includes two pads 332 separated from one another such that, when the surcingle 300 is disposed around the girth of the large animal 102, the pads 332 flank the spine of the large animal 102. In at least some embodiments, the one or more pads 332 are triangular-, or wedge-shaped.

In at least some embodiments, the surcingle 300 includes a control-module holder 342 for holding the control module 104. In which case, in at least some embodiments the first return wire 132a (see e.g., FIG. 3C) and the second return wire 132b are disposed within the body 306 of the first element 302 of the surcingle 300 and accessible for coupling to the control module 104 when the control module 104 is disposed in the holder 342, thereby electrically coupling the control module 104 to the return electrodes 208a and 208b.

As mentioned above, the one or more treatment electrodes can be disposed over the skin of the large animal 102 at the target stimulation location. Optionally, the target stimulation location is in proximity to an adversely-affected location on the large animal 102 for which therapy is sought. In at least some cases, the target stimulation location is located distally on the large animal 102 from the adversely-affected location on the large animal 102. For example, if the adversely-affected location is the knee of the large animal, the target stimulation location may be a location more distal on the adversely-affected forelimb than the adversely-affected location. In other cases, the target stimulation location can be located proximally on the large animal 102 from the adversely-affected location on the large animal 102.

FIG. 4A is a schematic side view of one embodiment of the system 100 disposed on a horse 402. The surcingle 300 is also disposed on the horse 402. The system 100 includes the control module 104 and the first stimulation circuit 201a. The first stimulation circuit 201a includes the first treatment lead 122a, the first treatment electrode 206a, the first return electrode 208a, and the first return wire 132a. The control module 104, the first return electrode 208a, and the first return wire 132a are disposed on the surcingle 300.

In FIG. 4A, the first treatment electrode 206a is shown disposed over a knee of the horse 402. During operation, therapeutic electrical signals output from the treatment electrode 206a are drawn to the first return electrode 208a, as shown by the dotted arrow 404. In at least some embodiments, a bandage wrap 406 is disposed over the first treatment electrode 206a to maintain the positioning of the treatment electrode 206a during operation. In at least some embodiments, the treatment electrode 206a includes adhesive to maintain positioning of the treatment electrode 206a in lieu of, or in addition to, using the wrap 406. In at least some embodiments, the first lead 122a is coiled to enable the horse 402 to move the stimulated forelimb freely without being constrained by the first lead 122a.

The treatment electrode 206a can be any suitable shape including for example, rectangular, circular, oval, triangular, diamond-shaped, cruciform-shaped, split-fingered, or the like. When the treatment electrode 206a is disposed over a bendable portion of the horse 402, such as a knee, it may be advantageous for the treatment electrode 206a to have a shape, such as a split-fingered shape that enables the horse 402 to bend the knee while the treatment electrode 206a is disposed over the knee without losing contact between the treatment electrode 206a and the skin of the horse 402.

In at least some embodiments, portions of the horse 402 over which the treatment electrode 206a and the return electrode 208a are disposed may be washed prior to application. In at least some embodiments, conductive gel may be used along an inner surface of the first treatment electrode 206a and the first return electrode 208a. The conductive gel may promote electrical conductivity between the skin of the horse 402 and the electrodes 206a and 208a.

In some cases, the system can be used to treat two or more adversely-affected locations on the large animal. FIG. 4B is a schematic side view of another embodiment of the system 100 disposed on the horse 402. In FIG. 4B, the system 100 includes each of the components shown in FIG. 4A plus the addition of the second stimulation circuit 201b. The second stimulation circuit 201b includes the second treatment lead 122b and the second treatment electrode 206b. In at least some embodiments, the second stimulation circuit 201b also includes the second return electrode 208b and the second return wire 132b (not shown in FIG. 4B, for clarity of illustration). In at least some embodiments, when the second stimulation circuit 201b includes the second return electrode 208b and the second return wire 132b, the second return electrode 208b and the second return wire 132b are disposed on the surcingle 300 (see e.g., FIGS. 3A-3C).

It will be understood that the system 100 can include any suitable number of stimulation circuits. FIG. 4C is a schematic side view of yet another embodiment of the system 100. In FIG. 4C, the system 100 is shown with four treatment electrodes 408a-d each coupled to the control module 104. The system 100 can include any suitable number of return electrodes. In FIG. 4C, the first return electrode 208a is shown. In at least some embodiments, the system 100 includes at least one additional return electrode disposed on other portions of the horse 402, such as the opposing side of the surcingle 300.

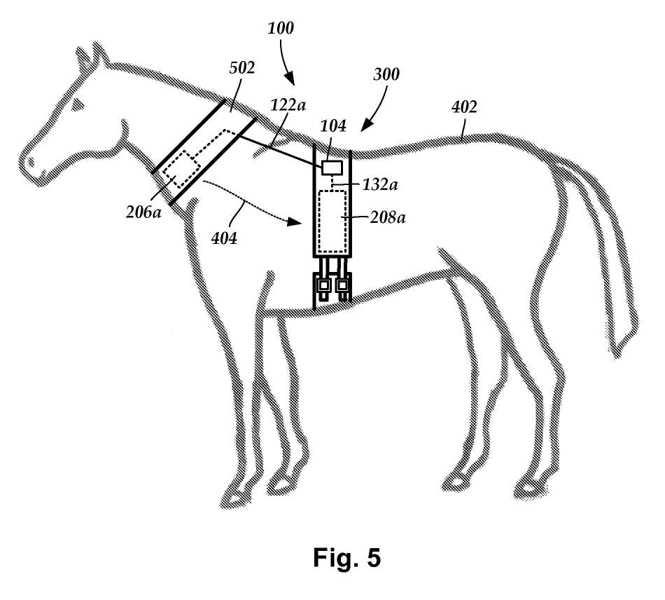

In FIGS. 4A-4C, the target stimulation locations were shown as including one or more of the horse's knees. It will be understood that the target stimulation location(s) can be any suitable location on the horse. FIG. 5 is a schematic side view of yet another embodiment of the system 100 shown in FIG. 4A. In FIG. 5, however, the treatment electrode 206a is disposed over the neck of the horse 402. The return electrode 208a is disposed on the surcingle 300. During operation, therapeutic electrical signals output from the treatment electrode 206a are drawn to the first return electrode 208a, as shown by the dotted arrow 404.

The treatment electrode 206a can be held in place against the target stimulation location of the horse 402 in any suitable manner including, for example, a wrap (see e.g., 406 in FIG. 4A), adhesive, a surcingle, a harness, a saddle, a crupper, a breeching, a breast collar, a head hood, a neck sleeve, a cover, a blanket, leggings, or the like or combinations thereof. In FIG. 5, the treatment electrode 206a is shown being held against the horse's neck by disposing the treatment electrode 206a under a neck sleeve 502.

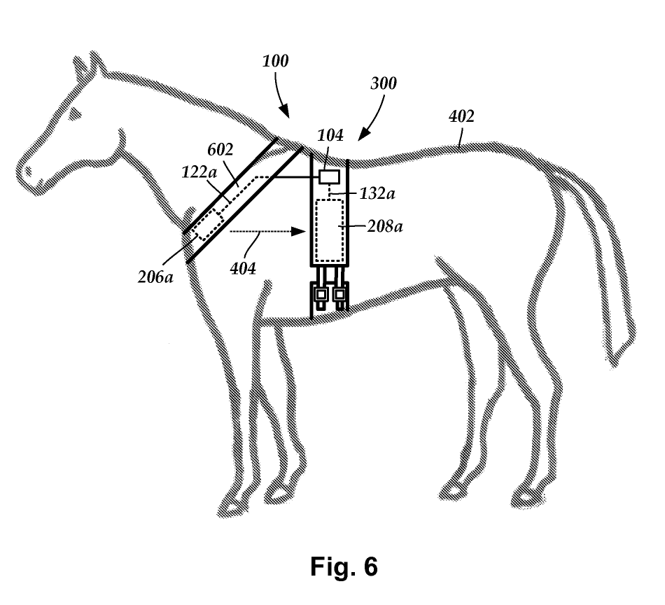

FIG. 6 is a schematic side view of yet another embodiment of the system 100 shown in FIG. 4A. In FIG. 6, the treatment electrode 206a is disposed over the upper forelimb of the horse 402. The return electrode 208a is disposed on the surcingle 300. During operation, therapeutic electrical signals output from the treatment electrode 206a are drawn to the first return electrode 208a, as shown by the dotted arrow 404. In FIG. 6, the treatment electrode 206a is shown being held against the horse's upper forelimb by disposing the treatment electrode 206a under a breast collar 602.

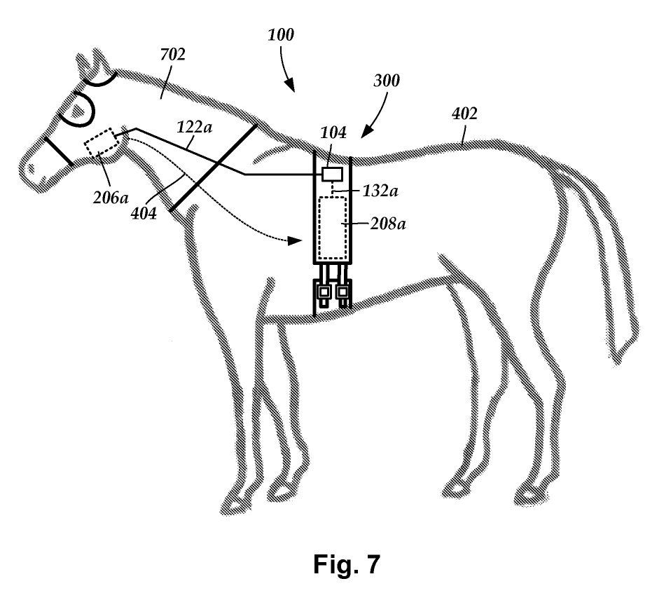

FIG. 7 is a schematic side view of yet another embodiment of the system 100 shown in FIG. 4A. In FIG. 7, the treatment electrode 206a is disposed over the jaw of the horse 402. The return electrode 208a is disposed on the surcingle 300. During operation, therapeutic electrical signals output from the treatment electrode 206a are drawn to the first return electrode 208a, as shown by the dotted arrow 404. In FIG. 7, the treatment electrode 206a is shown being held against the horse's neck by disposing the treatment electrode 206a under a head hood 702.

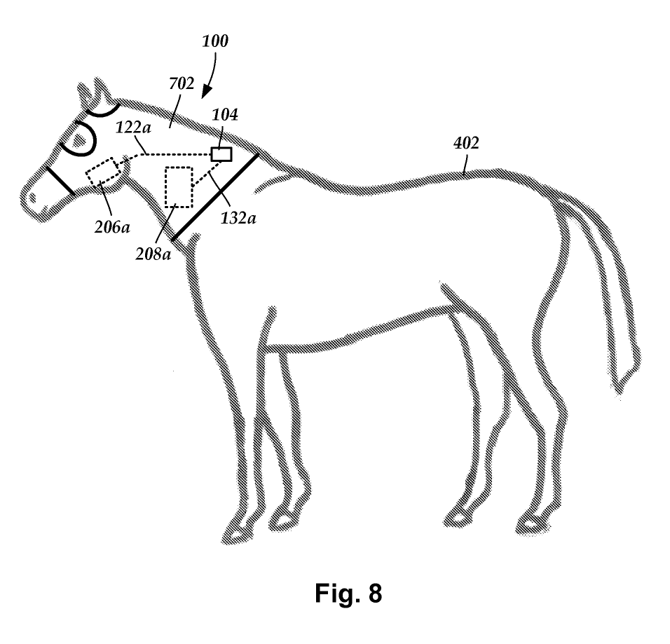

In FIGS. 4A-7, the control module and the return electrode(s) are shown disposed on the surcingle 300. It will be understood that the control module can be disposed at any suitable location on the horse 402. It will also be understood that the return electrode(s), likewise, can be disposed at any suitable location on the horse 402. FIG. 8 is a schematic side view of another embodiment of the system 100. In FIG. 8, the control module 104, the treatment electrode 206a, and the return electrode 208a are each disposed beneath the head hood 702 which, in turn, is disposed over the head of the horse 402.

Note that in FIGS. 7 and 8 the treatment electrode 206a is disposed in the same location. In FIG. 8, however, the return electrode 208a is disposed closer to the treatment electrode 206a than the return electrode 208a shown in FIG. 7. A reduction in the distance between the return electrode 208a and the treatment electrode 206a may reduce the distance that the electrical signals transmit through the horse 402.

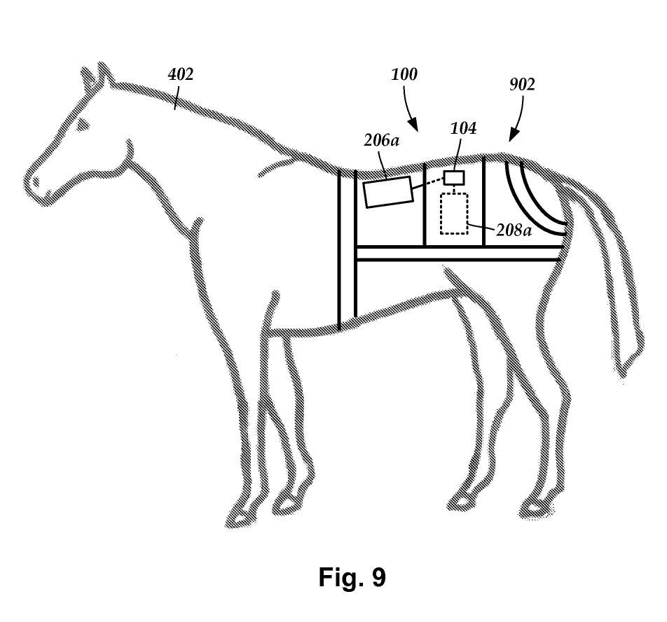

FIG. 9 is a schematic side view of another embodiment of the system 100. In FIG. 9, the control module 104 and the return electrode 208a are each disposed beneath a strap of a breeching 902 coupled to the horse 402. The treatment electrode 206a is disposed on the back of the horse 402. In FIG. 9, the treatment electrode 206a is coupled to the horse 402 using adhesive.

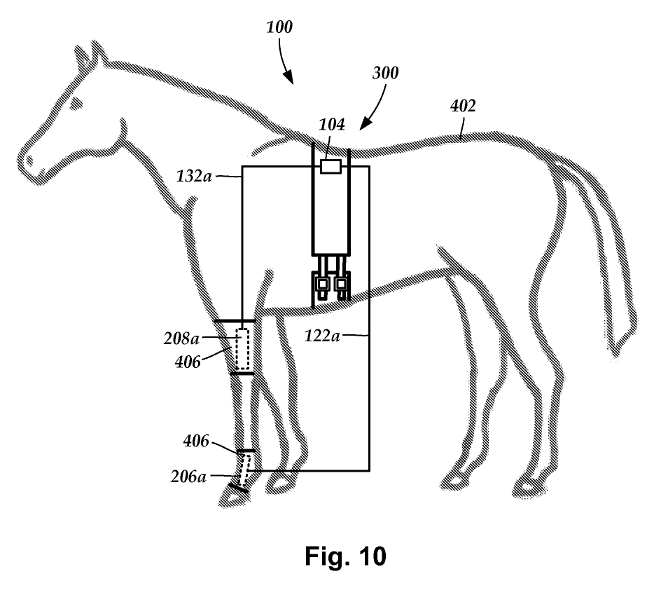

In at least some embodiments, the treatment electrode and the return electrode are both disposed on a forelimb (or hindlimb) of the horse. FIG. 10 is a schematic side view of another embodiment of the system 100. In FIG. 10, the treatment electrode 206a and the return electrode 208a are each disposed on a forelimb of the horse 402. The treatment electrode 206a is disposed over a fetlock of the horse 402. The control module 104 can be disposed in any suitable location on the horse 402. In FIG. 10, the control module 104 is disposed on the surcingle 300. The treatment electrode 206a and the return electrode 208a can be coupled to the horse 402 in any suitable manner. In FIG. 10, the treatment electrode 206a and the return electrode 208a are coupled to the horse 402 using wraps 406.

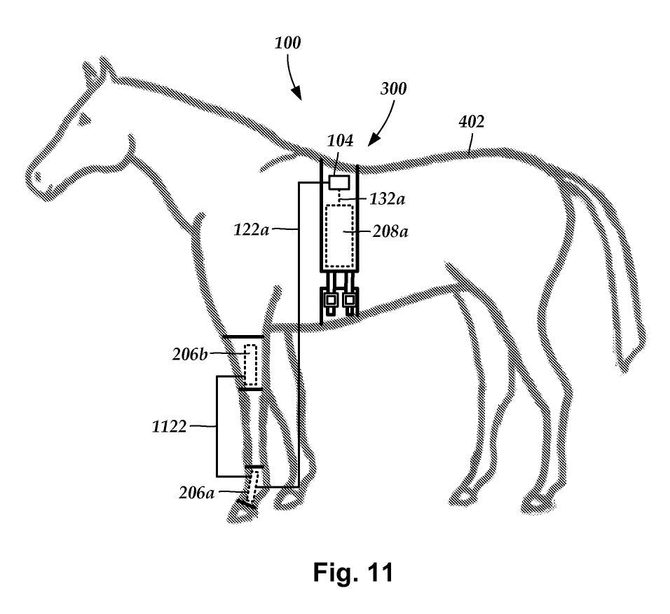

In at least some embodiments, the system includes one or more extension leads which enable one or more additional treatment electrodes to be added to the system by coupling the one or more additional treatment electrodes to the treatment electrode coupled to the control module via the lead 122a. FIG. 11 is a schematic side view of another embodiment of the system 100. In FIG. 11, the first treatment electrode 206a is disposed over a fetlock of the horse 402 and is coupled to the control module via the first lead 122a. The second treatment electrode 206b is disposed on another portion of the forelimb of the horse 402 and is coupled to the first treatment electrode 206a by an extension lead 1122. The control module 104 and the one or more return electrodes 208a can be disposed in any suitable location on the horse 402. In FIG. 11, the control module 104 and the return electrode 208a are shown disposed on the surcingle 300.

The above specification, examples and data provide a description of the manufacture and use of the composition of the invention. Since many embodiments of the invention can be made without departing from the spirit and scope of the invention, the invention also resides in the claims hereinafter appended.

What is claimed as new and desired to be protected by Letters Patent of the United States is:

1. An electrical stimulation system for providing non-invasive therapeutic stimulation to a large animal, the stimulation system comprising:
a surcingle configured and arranged for placement around a girth of the large animal, the surcingle comprising
an elongated first element having an inner surface, an outer surface, a first end, and an opposing second end, wherein the first element comprises a non-stretchable material, and
an elongated second element having a first end and an opposing second end, the first end of the second element coupleable to the first end of the first element and the second end of the second element coupleable to the second end of the first element, wherein the second element comprises a material that is significantly more stretchable than the material of the first element;
a plurality of foam wedges disposed on the inner surface of the first element, the plurality of foam wedges configured and arranged for disposing on either side of a spine of the large animal when the surcingle is disposed around the girth of the large animal;
a control module coupleable to the first element, the control module comprising a signal generator configured and arranged for generating electrical signals;
at least one lead having a proximal end and a distal end, the proximal end of the lead electrically coupleable to the control module;
at least one treatment electrode disposed at the distal end of the at least one lead, the at least one treatment electrode having a conductive inner surface configured and arranged for disposing over skin of the large animal at a target stimulation location, the at least one treatment electrode configured and arranged to transmit to the target stimulation location the electrical signals generated by the signal generator; and
at least one return electrode disposed on the inner surface of the first element, the at least one return electrode electrically coupled to the control module and configured and arranged to receive at least some of the electrical signals transmitted from the at least one treatment electrode.

2. The system of claim 1, wherein the signals generated by the signal generator are pulsed direct current signals.

3. The system of claim 1, wherein the at least one return electrode comprises a silver-impregnated material.

4. The system of claim 1, wherein the first end and the second end of the first element each comprises a plurality of first coupling elements and the first end and the second end of the second element each comprises a plurality of second coupling elements configured and arranged to mechanically mate with the plurality of first coupling elements.

5. The system of claim 4, wherein the first coupling elements comprise one of a plurality of buckles or a plurality of straps and the second coupling elements comprise the other of the plurality of buckles or the plurality of straps.

6. The system of claim 1, wherein the system comprises a plurality of treatment electrodes.

7. The system of claim 6, wherein the plurality of treatment electrodes comprises a first treatment electrode and a second treatment electrode, and wherein the first treatment electrode is coupled to the at least one lead.

8. The system of claim 7, further comprising an extension lead coupling the second treatment electrode to the first treatment electrode.

9. A method for providing therapeutic non-invasive electrical stimulation to a large animal, the method comprising:
providing the electrical stimulation system of claim 1;
disposing the surcingle of the electrical stimulation system around a girth of the large animal;
placing the at least one treatment electrode of the electrical stimulation system at one or more target stimulation locations on the skin of the large animal;
coupling the proximal end of the at least one lead of the electrical stimulation system to the control module of the electrical stimulation system;
coupling the distal end of the at least one lead to the at least one treatment electrode;
generating electrical signals by the signal generator of the electrical stimulation system;
outputting the electrical signals to the at least one treatment electrodes, the output electrical signal electrically stimulating the large animal in proximity to the at least one treatment electrode;
increasing an amplitude of the output electrical signals until a visible response to the electrical stimulation by the large animal is detected by a user of the electrical stimulation system, thereby establishing a sensory stimulation level; and
decreasing the amplitude of the output electrical signals to a sub-sensory stimulation level.

10. The method of claim 9, further comprising spreading conductive gel on one or more of the inner surface of the surcingle of the electrical stimulation system or the inner surface of the at least one treatment electrode of the electrical stimulation system.

11. The method of claim 9, wherein placing the at least one treatment electrode at one or more target stimulation locations on the skin of the large animal comprises placing the at least one treatment electrode on the neck of the large animal.

12. The method of claim 9, wherein placing the one or more treatment electrodes at one or more target stimulation locations on the skin of the large animal comprises placing the at least one treatment electrode on the jaw of the large animal.

13. The method of claim 9, wherein placing the one or more treatment electrodes at one or more target stimulation locations on the skin of the large animal placing the at least one treatment electrode on the back of the large animal.

14. The method of claim 9, wherein placing the one or more treatment electrodes at one or more target stimulation locations on the skin of the large animal comprises placing the at least one treatment electrode on at least one forelimb of the large animal.

15. The method of claim 9, wherein placing the one or more treatment electrodes at one or more target stimulation locations on the skin of the large animal comprises placing the at least one treatment electrode on at least one hindlimb of the large animal.

16. The method of claim 9, wherein placing the one or more treatment electrodes at one or more target stimulation locations on the skin of the large animal comprises placing the at least one treatment electrode on at least one knee of the large animal.

17. The method of claim 9, wherein placing the one or more treatment electrodes at one or more target stimulation locations on the skin of the large animal comprises placing the at least one treatment electrode on at least one fetlock of the large animal.

18. The method of claim 9, wherein placing the one or more treatment electrodes at one or more target stimulation locations on the skin of the large animal comprises placing the at least one treatment electrode on at least one hock of the large animal.

19. The method of claim 9, wherein placing the one or more treatment electrodes at one or more target stimulation locations on the skin of the large animal comprises placing the at least one treatment electrode on at least one stifle of the large animal.

20. The method of claim 9, wherein placing the one or more treatment electrodes at one or more target stimulation locations on the skin of the large animal comprises placing the at least one treatment electrode on at least one foot of the large animal.

21. The method of claim 9, wherein placing the one or more treatment electrodes at one or more target stimulation locations on the skin of the large animal comprises placing the at least one treatment electrode on at least one shin of the large animal.

22. The method of claim 9, wherein placing the one or more treatment electrodes at one or more target stimulation locations on the skin of the large animal comprises placing the at least one treatment electrode over at least one splint bone of the large animal.

23. The method of claim 9, wherein placing the one or more treatment electrodes at one or more target stimulation locations on the skin of the large animal comprises placing the at least one treatment electrode on at least one shoulder of the large animal.

24. An electrical stimulation system for providing therapeutic stimulation to a large animal, the stimulation system comprising:
a signal generator disposed in a control module, the signal generator configured and arranged to generate electrical signals;
a control-module holder configured and arranged for disposing on the large animal, the control-module holder configured and arranged for retaining the control module;
at least one coiled lead having a proximal end and a distal end, the proximal end of the lead electrically coupleable to the control module;
at least one treatment electrode disposed at the distal end of the at least one lead, the at least one treatment electrode having a conductive inner surface configured and arranged for disposing against skin of the large animal at a target stimulation location, the at least one treatment electrode configured and arranged to transmit to the target stimulation location the electrical signals generated by the signal generator;
at least one return wire having a proximal end and a distal end, the proximal end of the at least one return wire electrically coupled to the control module; and
at least one return electrode disposed at the distal end of the at least one return wire, the at least one return electrode having a conductive inner surface configured and arranged for disposing against skin of the large animal at a signal-receiving location that is remote from the target stimulation location, the at least one return electrode configured and arranged to receive at least some of the electrical signals transmitted to the target stimulation location and propagate those received electrical signals to the control module.

25. The system of claim 24, wherein the at least one return electrode is disposed on one of a harness, a head hood, a neck sleeve, a breast collar, a crupper, or a breeching.

26. The system of claim 24, wherein the control-module holder is disposed on one of a harness, a head hood, a neck sleeve, a breast collar, a crupper, or a breeching.
